(19) United States
(12) Patent Application Publication
(10) Pub. No.: US 2026/0308575 A1
(43) Pub. Date: Oct. 8, 2026

(54) BIOLOGICAL LIBRARIES AND METHODS OF PREPARING AND USING SAME

(71) Applicant: UNM RAINFOREST INNOVATIONS, Albuquerque, NM (US)

(72) Inventors: Rita E. Serda, Placitas, NM (US); Jimin GUO, Alburquerque, NM (US)

(21) Appl. No.: 19/715,071

(22) Filed: Jun. 22, 2026

Related U.S. Application Data
(63) Continuation of application No. 17/830,637, filed on Jun. 2, 2022, now abandoned.
(60) Provisional application No. 63/195,976, filed on Jun. 2, 2021.

(51) Int. Cl. C12N 15/10; A61K 35/12; C40B 40/02

(52) U.S. Cl. CPC C12N 15/1086; A61K 35/12; C40B 40/02; C12N 2330/31

(57) ABSTRACT

A biological library that includes a plurality of cryo-silicified cells, a plurality of dehydrated cryo-silicified cells, or both, where the cryo-silicified cells and/or the dehydrated cryo-silicified cells contain accessible biological information. In some embodiments, the biological information includes genetic information, proteomic information, or transcriptomic information. Members of the library may be analyzed to identify changes in the biological information associated with a medical condition or response to treatment. When the library includes B lymphocytes, cellular material from the B lymphocytes may be used to produce an antibody.

### CROSS-REFERENCE TO RELATED APPLICATION

This application claims the benefit of U.S. Provisional Patent Application No. 63/195,976, filed Jun. 2, 2021, which is incorporated herein by reference in its entirety.

### SUMMARY

This disclosure describes, in one aspect, a biological library that includes a plurality of cryo-silicified cells, a plurality of dehydrated cryo-silicified cells, or both. At least a portion of the cryo-silicified cells, the dehydrated cryo-silicified cells, or both comprise accessible biological information.

In some embodiments, the biological information comprises genetic information, proteomic information, or transcriptomic information.

In some embodiments, the plurality of cryo-silicified cells, a plurality of dehydrated cryo-silicified cells, or both are prepared from cells isolated from a patient infected with a virus, diagnosed as having a disease, or both.

In some embodiments, the cryo-silicified cells, dehydrated cryo-silicified cells, or both are prepared from tumor cells, B cells, tissue cells, or combinations thereof.

In another aspect, this disclosure describes a method for preserving a cell. Generally, the method includes obtaining a sample that includes a cell and cryo-silicifying the sample to create a cryo-silicified sample.

In some embodiments, the method further includes dehydrating the cryo-silicified sample to create a dehydrated cryo-silicified sample.

In some embodiments, the method further includes storing the cryo-silicified sample.

In some embodiments, the cryo-silicified sample includes a cellular material and the method further includes analyzing the cellular material. In some embodiments, the cellular material includes RNA, DNA, a protein, a fragment thereof, or combinations thereof. In some embodiments, the method further includes isolating the cellular material from the cryo-silicified sample. In some embodiments, the sample includes a cell infected with a pathogen. In some embodiments, the pathogen includes a virus having a viral genome, the cellular material includes RNA fragments of the transcribed viral genome, and the method further includes sequencing the RNA fragments and reconstructing the viral genome. In some embodiments, where the sample includes a B cell, the cellular material includes RNA, the method further incudes: making cDNA from the RNA; cloning the cDNA to into an expression vector; and expressing the expression vector to produce an immunoglobulin polypeptide encoded by the cDNA. In some embodiments, the method further includes administering the immunoglobulin polypeptide to a patient. In some embodiments, the cellular material includes at least a portion of a cellular genome and the at least a portion of the cellular genome is analyzed for a genetic mutation.

In another aspect, the present disclosure describes a method for analyzing differences between a first cellular material and a second cellular material. The method includes, obtaining a first cell from a subject, cryo-silicifying the first cell to create a cryo-silicified cell; storing the first cell; isolating a cellular material from the cryo-silicified firs cell; obtaining a second cell; isolating a second cellular material from the second cell; and analyzing the first cellular material and the second cellular material for a difference between the first cellular material and the second cellular material.

In some embodiments, the method further includes dehydrating the cryo-silicified cell to create a dehydrated cryo-silicified cell and rehydrating the dehydrated cryo-silicified cell.

In some embodiments, the first cell is a tumor cell, the second cell is normal reference cell, and the difference between the first cellular material and the second cellular material indicates the subject's response to a selected therapy.

In some embodiments, where the first cell is obtained from a subject inflicted with a medical condition at a first time; the second cell is obtained from the subject at a second time point occurring at a predetermined time after a therapy is administered to the subject; the step of analyzing the first cellular material and the second cellular material for a difference between the first cellular material and the second cellular material includes identifying a change in the second cellular material that is indicative of the medical condition compared to the first cellular material that is indicative of the medical condition.

In some embodiments, the step of analyzing the cellular material of the second cell further includes cryo-silicifying the second cell to create a second cryo-silicified cell; and storing the second cryo-silicified cell.

The above summary is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1. Electrophoretic analysis of RNA integrity of silicified and silicified and dehydrated RNA samples after storage for more than one year. Lane 1: size ladder; Lane 2: silicified(S) RNA sample 1; Lane 3: silicified(S) RNA sample 2; Lane 4: silicified(S) and dehydrated (D) RNA sample 1; Lane 5: silicified(S) and dehydrated (D) RNA sample 2. All four RNA samples contained intact 18S and 28S ribosomal RNA bands.

FIG. 2. Electropherogram of ribosomal RNA peaks for silicified RNA samples. Both silicified samples have intact 18S and 28S ribosomal RNA peaks with no shift and no signs of peak degradation.

FIG. 3. Electropherogram of ribosomal RNA peaks for silicified and dehydrated RNA samples. Both silicified/dehydrated samples have intact 18S and 28S ribosomal RNA peaks with no shift and no signs of peak degradation.

FIG. 4. Complete data for silicified RNA sample 2, showing an RNA Integrity Number (RIN) of 9.9, a 28S/18S rRNA ratio of 2.3, and a concentration of 378 ng/μl.

FIG. 5. Complete data for silicified and dehydrated RNA sample 2, showing an RNA Integrity Number (RIN) of 8.4, a 28S/18S rRNA ratio of 3.0, and a concentration of 764 ng/μl.

FIG. 6. Agarose gel showing the RT-PCR products from fresh ID8 cells (lanes 2 and 5), silicified BR5-Akt ovarian cancer cells (lanes 3 and 6), and dehydrated cryo-silicified BR5-Akt ovarian cancer cells (lanes 4 and 7) using random hexamer reverse primers (lanes 2-4) or a BRCA1 reverse primer (lanes 5-7).

### DETAILED DESCRIPTION OF ILLUSTRATIVE EMBODIMENTS

Efforts to rapidly develop diagnostics and/or therapeutics often involve study of cell and/or tissues from patients. Cells of patients contain biological information in the form of, for example, genetic information, proteomic information, and transcriptomic information.

Currently, most tissue repositories use formalin fixation and paraffin embedding to preserve patient tissues or cells. The fixation process and the process by which tissues are embedded in paraffin may result in difficult extraction of RNA from the cells and/or RNA that is degraded and/or chemically modified (Xiao et al., 2013, J Pathol 228(4):535-545). An alternative method to retain high quality RNA is flash freezing of tissue in liquid nitrogen or on dry ice. Upon flash freezing, the frozen sample requires continual storage at −80° C. or below, creating problems when freezing facilities are not available or sample collection is decentralized, and samples need to be transported at cryogenic temperatures.

This disclosure describes a process involving cryo-silicification to preserve a sample that includes cells and/or tissues. As used herein, the term “sample” referees to a cell, plurality of cells, a tissue, or combinations thereof. The cryo-silicification process for preserving samples avoids aldehyde fixation, which can chemically modify RNA in the cells, and avoids the need to embed cells and/or tissues in paraffin. In some embodiments, the cryo-silicified samples may undergo subsequent dehydration to from dehydrated cryo-silicified samples. Cryo-silicification, with or without a separate dehydration step, enables dry, long-term storage of cells and tissues so that the cells or tissues can be rehydrated, and the rehydrated, intact cells can be used as a source of preserved biological information.

As used herein, the term “silicification” is used to refer to the process of exposing a sample to silicon containing compound that has silicon and oxygen covalently bonded to the silicon atom. In some embodiments, the silicon containing compounds is an orthosilicate. As used herein, “orthosilicate” refers to a compound that includes the formula of SiO₄. As such, as used herein, “orthosilicate” includes silicic acid (Si(OH)₄); the SiO₄ anion; salts of the SiO₄ anion (SiO₄⁻⁴); esters (or sometimes called ethers) of SiO₄, for example Si(OR)₄ where each R is independently alkyl; hydrates thereof, and derivatives thereof. Examples of esters of silicic acid include, but are not limited to, tetramethyl orthosilicate (tetramethoxysilane) and tetraethyl orthosilicate (tetraethoxysilane). The term “silicified cell,” “silicified tissue,” and “silicified sample” refer to a cell, tissue, or sample that has undergone silicification.

As used herein, the terms “cryo-silicification” or “cryo silicification” refer to the process of silicification of sample where the silicification process occurs at room temperature for a period of time followed by subsequent incubation of the sample in an environment with a temperature of 0° C. to 80° C. for a period of time. In cryo-silicification, silicification of the sample occurs both when the sample is incubated at room temperature and when the sample is incubated at a temperature of 0° C. to 80° C. In cryo-silicification, the sample is exposed to the silicon-containing compound when incubated at ambient temperature and when incubated at a temperature of 0° C. to 80° C. In some embodiments, the cryo-silicified sample is thawed. In such embodiments, silicification further occurs during the thawing. The terms “cryo-silicified cell,” “cryo-silicified tissue,” and “cryo-silicified sample” refer to a cell, tissue, or sample that has undergone cryo-silicification.

Samples that are cryo-silicified may be undissociated, for example, as a tissue sample or other multicellular structure. Samples that are cryo-silicified may include dissociated cells. Thus, as used herein and unless otherwise specified in the context of the description, reference to a cryo-silicified sample and or cell includes either a dissociated cell, an undissociated plurality of cells, or a mixture of dissociated and undissociated cells. Exemplary methods for silicifying cells and cryo-silicifying cells or tissues are described in International Publication No. WO 2019/055620 (PCT/US2018/050831 ), U.S. Patent Application Publication No. US 2020/0276286 A1, and International Publication No. WO 2020/185449 A1 (PCT/US2020/020776).

The sample subjected to cryo-silicification and/or cryo-silicification followed by dehydration can be any sample containing or suspected of containing biological information relevant for later analysis. For example, B cells may be cryo-silicified and stored for later analysis of antibodies produced by the B cells. As another example, cells known to be infection targets of an infectious agent of interest (e.g., a virus or bacterium) may be cryo-silicified and/or cryo-silicified and dehydrated, and stored for later analysis of the infectious agent (e.g., reconstructing the genome of the infectious agent). As another example, tumor cells from a patient may be cryo-silicified and/or cryo-silicified and dehydrated, and stored for later analysis of differences between the biological information in the tumor cell versus the patient's normal cells.

As used herein, the term “dehydration” refers to the process of dehydrating a cell or a tissue. As such, the terms “dehydrated cell,” “dehydrated tissue,” and “dehydrated sample” refer to a cell, a tissue, or a sample that has undergone dehydration. A dehydrated sample is a sample that has 15 wt-% or less of water content based on the total cell or tissue mass. In some embodiments, the dehydrated sample has a 15 wt-% or less, 10 wt-% or less, 5 wt-% or less, or 1 wt-% or less of water content based on the total sample. In embodiments, a cryo-silicified sample may be dehydrated while it is frozen. In some embodiments, a cryo-silicified cell may first be thawed and then subsequently dehydrated.

As used herein, the terms “dehydrated cryo-silicified,” cryo-silicified dehydrated,” “dehydrated cryo silicified,” and “cryo silicified dehydrated” are used interchangeably and refer to a sample that has undergone cryo-silicification and dehydration. As such, sample is both cryo-silicified and dehydrated.

Cryo-silicification and/or cryo-silicification and subsequent dehydration allows one to reconstitute the cryo-silicified and/or dehydrated cryo-silicified samples to perform one or more analyses on biological information contained within the cells. Exemplary biological information extractable from reconstituted cells following cryo-silicification and/or cryo-silicification and subsequent dehydration include, but is not limited to, genetic information (e.g., from DNA or certain classes of RNA from the reconstituted cell), proteomic information (e.g., from protein of the reconstituted cell), or transcriptomic information (e.g., from mRNA of the reconstituted cell).

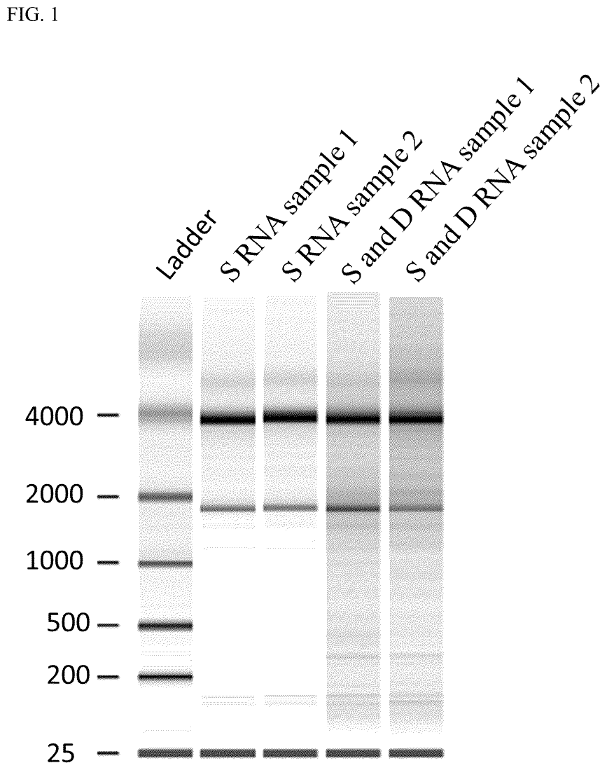

FIG. 1 shows data indicating that cryo-silicified cells, whether subsequently dehydrated (dehydrated cryo-silicified cells) or not, stored at −80° C. for more than one year retained integrity of RNA extracted from the cells following thawing and reconstitution. Electrophoretic analysis showed that two cryo-silicified samples and two samples subjected to cryo-silicification and dehydration all contained intact 18S (~2000 nucleotides) and 28S (~4000 nucleotides) ribosomal RNA bands.

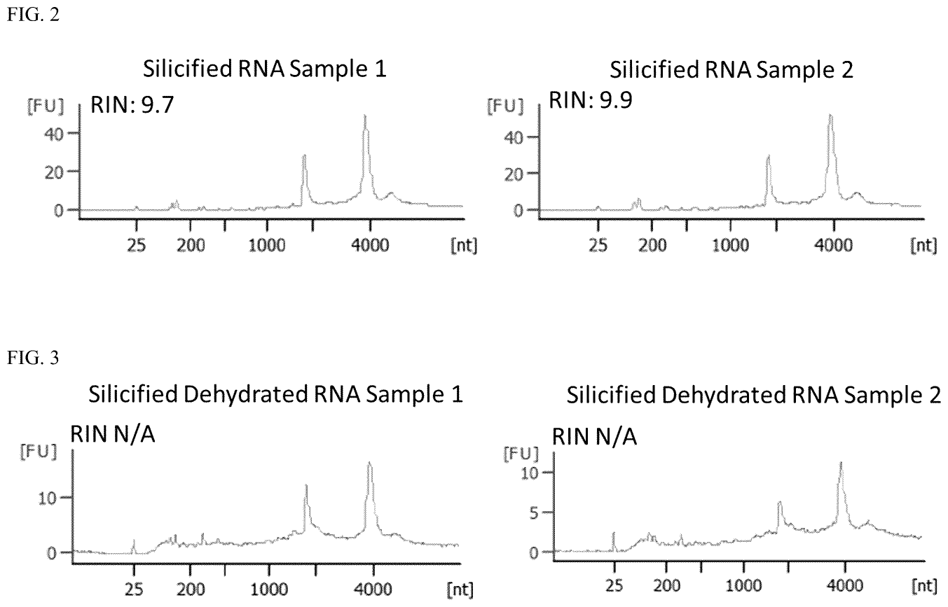

FIG. 2 shows electropherograms of RNA extracted from two different cryo-silicified cell samples. FIG. 3 shows similar data for RNA extracted from the same cryo-silicified cell samples as shown in FIG. 2, but the cryo-silicified cells were dehydrated following cryo-silicification. The samples were desiccated overnight in an electronic desiccator at room temperature. All four RNA samples contained an intact 18S ribosomal RNA peak and an intact 28S ribosomal RNA peak with no peak shift or any sign of peak degradation. The silicified cells had RNA Integrity Numbers (RIN) of 9.7 (Silicified RNA 1) and 9.9 (Silicified RNA 2), where 10 is perfect RNA. Debris in the silicified/dehydrated samples interfered with RIN determination.

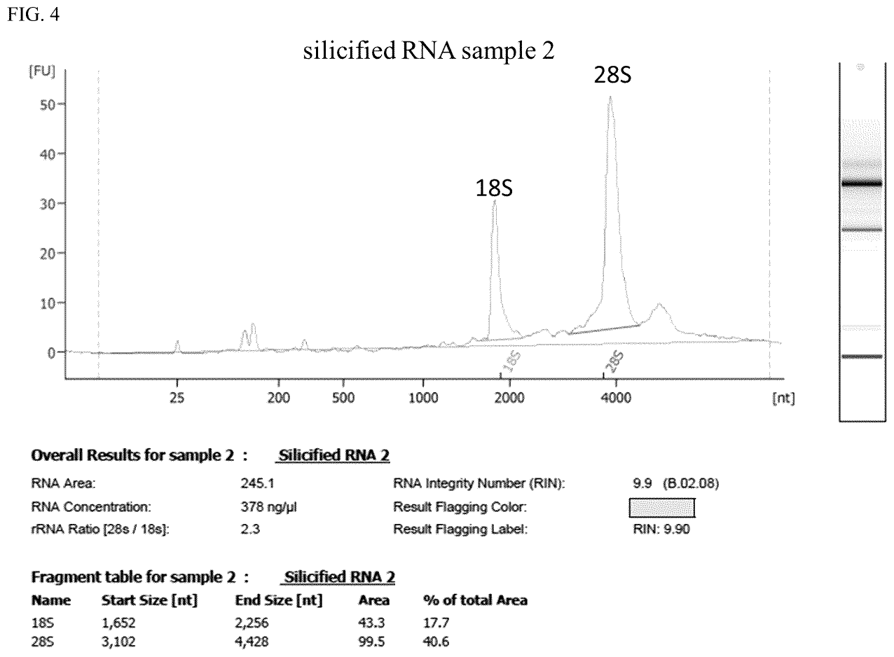

FIG. 4 shows additional data for the Silicified RNA 2 sample. The rRNA ratio (28S/18S) was 2.3 with a concentration of 378 ng/μl.

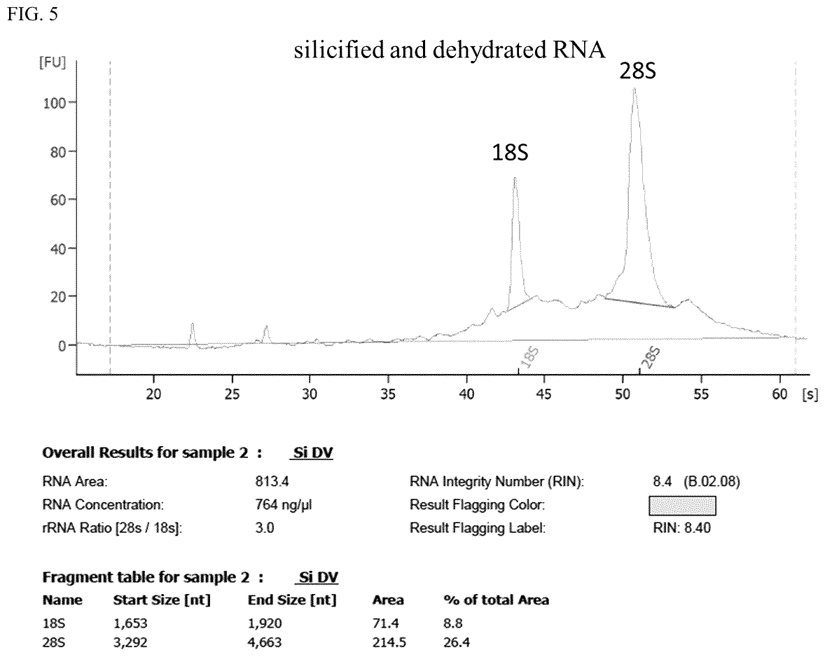

A unique cryo-silicified cell sample that was stored at −80° C. for 14 months was dehydrated, followed by room temperature storage for 24 hours in an electronic desiccator. The electropherogram of the dehydrated cryo-silicified cell sample presented in FIG. 5 shows a RIN score of 8.4 for RNA recovered from this sample. The rRNA ratio (28S/18S) was 3.0 with a concentration of 764 ng/μl.

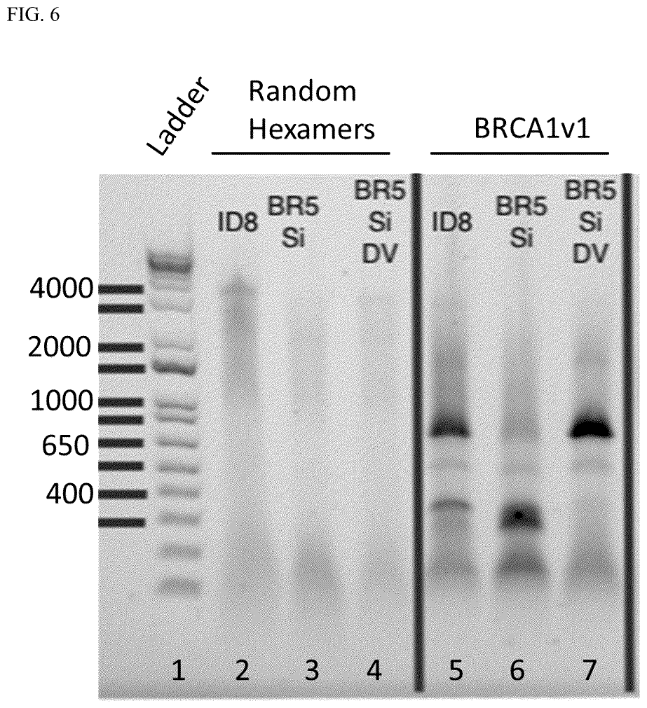

Biological information can be extracted from reconstituted cells. For example, RNA was extracted from fresh cells, cryo-silicified cells, and dehydrated cryo-silicified cells The extracted RNA was reverse transcribed and amplified via a reverse transcription polymerase chain reaction (RT-PCR) using random hexamer reverse primers, or a BRCA1 specific reverse primer. The gel shown in FIG. 6 indicates that RNA isolated from both cryo-silicified cells and dehydrated cryo-silicified cells can be used for gene amplification.

The cryo-silicified samples and dehydrated cryo-silicified samples therefore represent a preserved library of accessible biological information contained within the cells, whether dissociated or undissociated, subjected to cryo-silicification and/or dehydration.

Thus, in one aspect, this disclosure describes a library composition that includes a plurality of cryo-silicified samples that possess accessible biological information. In another aspect, this disclosure describes a library composition that includes a plurality of dehydrated cryo-silicified samples that possess accessible biological information. In some embodiments, the library includes both cryo-silicified and dehydrated cryo-silicified samples that each possess accessible biological information.

As used herein, the term “accessible biological information” refers to biological information that is extractable from a cell. Extractable biological information includes, but is not limited to, RNA, DNA, proteins, lipids, sugars, fragments thereof, and combinations thereof.

In some embodiments, the accessible biological information may be genetic information (e.g., from extracted DNA or RNA), transcriptomic information (e.g., from extracted mRNA), proteomic information (e.g., from extracted protein), or other biological information accessible from extracted biomolecules from the sample. In some cases, the biological information may be endogenous—i.e., originating from cells of the organism from which the cell sample subjected to cryo-silicification and/or dehydration is obtained. In other embodiments, the biological information may be exogenous—e.g., originating from, for example, a virus or bacterium that had infected cells of the organism from which the cell sample subjected to cryo-silicification and/or dehydration is obtained.

In some embodiments, a sample that includes a plurality of cells, cells within the plurality of cells may be dissociated from other cells prior to cryo-silicification or cryo-silicification and subsequent dehydration. In other embodiments, the plurality of cells may be cryo-silicified or cryo-silicified and subsequently dehydrated while still associated with other cells, as may be the case if a multicellular tissue is subjected to cryo-silicification or cryo-silicification and subsequent dehydration.

A sample obtained from any source may be subjected to cryo-silicification or cryo-silicification and subsequent dehydration. In some embodiments, the sample source is a tissue such as a connective tissue, nervous tissues, skeletal muscle tissue, cardiac muscle tissue, epithelial tissue, smooth muscle tissue, cartilage tissue, or combinations thereof. Other sample sources that may be cryo-silicified for preservation include, but are not limited to, bone marrow, cerebrospinal fluid, ascites, blood, urine, semen, synovial fluid, pericardial fluid, amniotic, saliva, nasal fluid, gastric fluid, breast milk, or pleural fluid. In some embodiments, the cell source is a human cell source.

A sample that includes any suitable cell type or cell tissue may be subjected to cryo-silicification or cryo-silicification and subsequent dehydration. Example cell types include, but are not limited to, embryonic stem cells, adult stem cells, erythrocytes, neurons, skeletal muscle cells, cardiac muscle cells, smooth muscle cells, chondrocytes, osteoblasts, osteoclasts, osteocytes, lining cells, keratinocytes, melanocytes, Merkel cells, Langerhans cells, endothelial cells, epithelial cells, white adipocytes, brown adipocytes, spermatozoa, and ova.

Preserved samples that include immune cells may be of interest for future analysis. As such, In some embodiments the library includes cryo-silicified and/or dehydrated cryo-silicified samples that include immune cells. Example immune cells include lymphocytes, monocytes, macrophages, basophils, neutrophils, and eosinophils. Specific lymphocytes include T-lymphocytes, B-lymphocytes, and natural killer (NK) cells. T-lymphocytes provide antigen-specific immune responses. Example T-cells include memory T-cells, cytotoxic T-cells, helper T-cells, and suppressor T-cells. B-lymphocytes are involved in humoral immunity by producing antibodies. Example B-cells include plasma cells and memory B-cells.

Thus, in some embodiments, the sample subjected to cryo-silicification may be obtained from a subject having or suspected of having a disease. As used herein, the term “at risk” refers to a subject that may or may not actually possess the described risk. Thus, for example, a subject “at risk” of infectious condition is a subject present in an area where other individuals have been identified as having the infectious condition and/or is likely to be exposed to the infectious agent even if the subject has not yet manifested any detectable indication of infection by the infectious agent and regardless of whether the subject may harbor a subclinical amount of the infectious agent. As another example, a subject “at risk” of a non-infectious condition is a subject possessing one or more risk factors associated with the condition such as, for example, genetic predisposition, ancestry, age, sex, geographical location, lifestyle, or medical history. In some embodiments, the subject is a human.

Thus, the library can include cryo-silicified samples and/or dehydrated cryo-silicified samples, that include cells isolated from a subject infected with an infectious agent such as, for example, a virus or bacterium. In other embodiments, the library can include cryo-silicified samples and/or dehydrated cryo-silicified samples, whether prepared from immune cells such as, for example, B cells. In other embodiments, the library can include cryo-silicified samples and/or dehydrated cryo-silicified samples prepared from tumor cells. Samples could be collected longitudinally from the same subject to show disease or treatment-induced changes, or from unique subjects. Other candidate cell types include but are not limited to embryonic cells, stem cells, macrophages, dendritic cells, T cells, NK cells, neutrophils, hepatocytes, nerve cells, sperm, egg cells, skin, or muscle cells.

In another aspect, this disclosure describes a method for cryo-silicifying a sample to create a cryo-silicified sample. The method includes, obtaining a sample and cryo-silicifying the sample. The sample may be obtained from any source and/or may include any cell or tissue as described herein.

Cryo-silicifying the sample includes silicifying the sample for a first period of time at a first temperature and for a second period of time at a second temperature to create a cryo-silicified sample. Silicifying includes exposing the sample to a silicon containing compound creating a silicified sample. In some embodiments, the silicon containing compound is silicic acid, tetramethyl orthosilicate, tetraethyl orthosilicate, salts thereof, hydrates thereof, or combinations thereof. In some embodiments, the sample is exposed to a silicon containing compound solution. In some embodiments, the silicon containing compound solution is an aqueous solution. In some embodiments, when the silicon containing compound is silicic acid, the silicic acid may be made in situ from a precursor compound.

In some embodiments, additional components may be added to the silicon containing compound solution. The addition components may be a buffer agent, a salt, an acid, other preservatives, and combinations thereof. Example buffer agents include, but are not limited to, 2-(N-morpholino)ethanesulfonic acid; tris(hydroxymethyl)aminomethane; 1,3-bis(tris(hydroxymethyl)methylamino)propane; piperazine-N,N′-bis(2-ethanesulfonic acid); 2-Hydroxy-3-morpholinopropanesulfonic acid; 4-(2-hydroxyethyl)-1-piperazineethanesulfonic acid; N-cyclohexyl-3-aminopropanesulfonic acid; N-(2-Hydroxyethyl)piperazine-N′-(4-butanesulfonic acid; tricine; phosphate; triethanolamine; glycinamide; 2,2′,2″-Nitrilotriacetic acid; N-(2-Acetamido)-2-aminoethanesulfonic acid; 2-aminoethyl(trimethyl)azanium;chloride;hydrochloride; 3-(Morpholin-4-yl)propane-1-sulfonic acid; 3-[[1,3-dihydroxy-2-(hydroxymethyl)propan-2-yl]amino]-2-hydroxypropane-1-sulfonic acid; 3-[4-(2-Hydroxyethyl)piperazin-1-yl]propane-1-sulfonic acid; tris(hydroxymethyl)methylamino]propanesulfonic acid; acetate; and combinations thereof.

Examples of salts that may be added to the silicon containing compound solution include, but are not limited to, sodium salts such as sodium chloride, potassium salts, acetate salts, carbonate salts, ammonium salts, citrate salts, and the like.

In some embodiments, acids such as hydrochloric acid may be added to the silicon containing compound solution to adjust the pH of the solution. In some embodiments, the pH of the solution is 7 or less, 6 or less, 5 or less, 4 or less, 3 or less, or 2 or less.

In some embodiments, silicifying a sample includes exposing the sample to the silicon containing compound and/or solution at a first temperature for a first incubation time. In some embodiments the first incubation time is two minutes or more, five minutes or more, 10 minutes or more, 30 minutes or more, or one hour or more, or 24 hours or more. In some embodiments, the first incubation time is five minutes to ten minutes.

In some embodiments, the first incubation is performed at ambient temperature. In some embodiments, the first incubation is performed at a temperature below ambient temperature. In some embodiments, the first incubation is performed at a temperature above ambient temperature. The first temperature at which incubation is performed may inform the length of the first incubation time.

Cryo-silicifying the sample includes freezing the sample during the silicification process. As such, cryo-silicifying the sample includes incubating the sample at a second temperature for a second incubation time during the silicification process. In some embodiments, the second temperature is a temperature designed to freeze the sample. As such, the second temperature may be referred to as the freezing temperature. As used herein, “freezing” includes exposing the sample to a freezing temperature where the freezing temperature is a temperature that is below the freezing point of water at a given atmospheric pressure. The sample may be frozen using any suitable technique. In some embodiments, the sample may be frozen by placing in a freezer. In some embodiments, the interior of the freezer is at −80° C., −20° C., 0° C., or any temperature between −80° C. and 0° C., any temperature that is colder than −80° C. In some embodiments, the sample may be frozen by exposing the sample to liquid nitrogen or dry ice.

In some embodiments, the method further includes dehydrating the cryo-silicified sample to create a dehydrated cryo-silicified sample. Dehydrating the cryo-silicified sample includes removing water from the cryo-silicified sample to create a dehydrated cryo-silicified sample. In some embodiments, a frozen cryo-silicified cell may be dehydrated. In some embodiments, a frozen cryo-silicified cell is thawed and then is subsequently dehydrated. Thawing refers to the process of liquifying frozen liquid (e.g., water) that is within a sample. In some embodiments, a frozen cryo-silicified sample may undergo dehydration after being completely thawed, that is, all of the frozen liquid in the sample has been liquefied. In some embodiments, a frozen cryo-silicified sample may undergo dehydration after being partially thawed, that is, some but not all of the frozen liquid in the sample has been liquefied.

Any suitable dehydration method for removing water may be used. For example, the sample may be exposed to an elevated temperature above ambient temperature (e.g., in an oven), thereby evaporating the water from the sample. In another example, the sample may be dehydrated using under vacuum at room temperature. In another example, the sample may be dehydrated under vacuum at a temperature that is higher than room temperature. In yet another example, the sample may be dehydrated by freezing the sample, or subjecting the already frozen cryo-silicified sample, to a low pressure environment thereby removing the water via sublimation (e.g., using a lyophilizer). The length of time the cryo-silicified sample undergoes the dehydration process is dependent on the dehydration method and the desired final water content.

In some embodiments, the method further includes storing the cryo-silicified cell sample and/or the dehydrated cryo-silicified sample for a storage time. In some embodiments, the cryo-silicified sample and/or dehydrated cryo-silicified sample are stored frozen at a temperature of −80° C., −20° C., 0° C., or any temperature between −80° C. and 0° C., any temperature that is colder than-80° C. In some embodiments, the dehydrated cryo-silicified samples are stored at temperatures greater than freezing. In some embodiments, the cryo-silicified cell sample and/or the dehydrated cryo-silicified sample are stored at 4° C. In some embodiments, the cryo-silicified cell sample and/or the dehydrated cryo-silicified sample are stored at ambient temperature. In some embodiments, the cryo-silicified cell sample and/or the dehydrated cryo-silicified sample are stored in a desiccator or under vacuum.

The cryo-silicified cell sample and/or the dehydrated cryo-silicified sample may be stored for a storage time. In some embodiments, the storage time is 1 hour or greater, 1 day or greater, 1 year or greater, 10 years or greater, or 100 years or greater.

In another aspect, this disclosure describes a method for analyzing molecular content of a cryo-silicified sample and/or a dehydrated cryo-silicified sample. The cryo-silicified sample and/or dehydrated cryo-silicified sample may be any sample as described elsewhere herein. The cryo-silicified sample and/or dehydrated cryo-silicified sample may have been prepared and/or stored described elsewhere herein. Generally, the method includes isolating at least one cellular material from the cryo-silicified cell sample and/or the dehydrated cryo-silicified sample and analyzing the cellular material. In some embodiments, the cryo-silicified sample or dehydrated cryo-silicified sample can be obtained from a library that includes cryo-silicified cells and/or dehydrated cryo-silicified cells.

Cellular material includes any material that includes biological material. Examples of cellular material include, but are not limited to, RNA, DNA, a protein, lipid, and other cellular materials that include biological information, or a fragment of any of the foregoing. In some embodiments in which RNA is analyzed, the RNA can include mRNA.

In some embodiments, the cellular material being analyzed may be exogenous—e.g., transcribed or otherwise originating from the genome, transcriptome, or proteome of an infectious agent that had infected the cell that was cryo-silicified. When the cellular material being analyzed is exogenous, the information contained within the cellular material may allow one to reconstruct the genome, transcriptome, or proteome of the infectious agent.

In other embodiments, the cellular material being analyzed can be endogenous—e.g., transcribed or otherwise originating from the genome, transcriptome, or proteome of the cell that was cryo-silicified and/or cryo-silicified and dehydrated. For example, the cellular material being analyzed may be the cellular genome of the cell subjected to cryo-silicification and/or cryo-silicification and dehydration. In such embodiments, the cellular genome, or a portion of the cellular genome, may be analyzed, for example, to detect genetic mutations. In other cases, the cellular material being analyzed may be the cellular transcriptome of the cell subjected to cryo-silicification or cryo-silicification and dehydration. In such embodiments, the cellular transcriptome may be analyzed, for example, to determine gene expression.

In some embodiments, the cryo-silicified cell and/or dehydrated cryo-silicified cell may be prepared from a B cell. In such embodiments, the cellular material being analyzed can include mRNA of the B cell. In some of these embodiments, the method can further include making a cDNA from the mRNA extracted from the B cell. In some embodiments, the mRNA may encode an immunoglobulin polypeptide (e.g., an antibody) of interest for use in binding to a specific antigen to treat a condition. Methods for making cDNA from mRNA are known and generally include reverse transcription. The mRNA and/or the cDNA may be amplified, for example, using a polymerase chain reaction (PCR). Once made, the cDNA may then, if desired, be cloned into an expression vector so that a polypeptide (e.g., an antibody) encoded by the cDNA can be translated and expressed.

In some embodiments where the cellular material being analyzed is from a dehydrated cryo-silicified sample, the method may include rehydrating the dehydrated cryo-silicified sample. As used herein “rehydration” refers to the process of re-introducing water into the sample. A “rehydrated” sample is a sample that has undergone rehydration. A rehydrated sample has regained 50% or greater of the water content it had prior to dehydration. Any suitable method of rehydration may be used. For example, a dehydrated cryo-silicified sample may be directly exposed to, or suspended in, the aqueous solution that will be used for isolating cellular material and/or analyzing cellular material. In another example, rehydration involves exposing a dehydrated cryo-silicified cell to an alcohol: aqueous solution mixture step down gradient where in each step the amount of alcohol is decreased and the amount of the aqueous solution is increased. In this method, the dehydrated cryo-silicified cell is exposed to 100% alcohol for a period of time, 95%:5% alcohol: aqueous solution for a period of time; 90%:10% alcohol:aqueous solution for a period of time; 80%:20% alcohol:aqueous solution for a period of time; 70%:30% alcohol: aqueous solution for a period of time; 60%:40% alcohol: aqueous solution for a period of time; 50%:50% alcohol: aqueous solution for a period of time; and 100% aqueous solution. The aqueous solution may be the solution that will be used in isolating cellular material and/or analyzing cellular material.

Methods for isolating cellular material from a sample are known. For example, a general method for isolating DNA and/or RNA includes, lysing the cell, loading the lysate onto a column where the DNA and/or RNA is isolated on the column, washing to column to remove debris, and eluting the DNA and/or RNA from the column. If desired, the isolated DNA and/or RNA may be further purified using for example, ethanol precipitation, phenol-chloroform extraction, or column chromatography. Isolated DNA and/or RNA may be concentrated, for example, using ethanol precipitation. Methods for isolating proteins include, but are not limited to, column chromatography, antibody pull down assays, and gel electrophoresis.

The cellular material may be analyzed using any method suitable for analyzing the cellular material being analyzed. Thus, methods used to analyze the cellular material include, but are not limited to, mass spectrometry, electrophoresis, RNAseq, DNAseq, chromatography, a DNA microarray assay, an RNA microarray assay, immunoprecipitation assays, co-immunoprecipitation assays, immunoassays such as ELISA (enzyme-linked immunosorbent assay), or high-throughput sequencing. In some embodiments, cellular material extracted from a cryo-silicified sample and/or dehydrated cryo-silicified sample can be subjected to more than one analytical method.

In another aspect, this disclosure describes a method of preparing an antibody that binds to an antigen associated with a condition. Generally, the method includes obtaining a B cell from a subject having the condition, cryo-silicifying the B cell to create a cryo-silicified cell or cryo-silicifying and dehydrating the B cell to form a dehydrated cryo-silicified cell, isolating a polynucleotide that encodes at least a portion of an immunoglobulin, cloning the polynucleotide into an expression vector, expressing the polynucleotide to produce an immunoglobulin polypeptide, and screening the immunoglobulin to determine whether the immunoglobulin polypeptide specifically binds the antigen. In some embodiments, where the B cell was cryo-silicified and dehydrated, the method may further include rehydrating the dehydrated cryo-silicified cell as described elsewhere herein. The polynucleotide that encodes the immunoglobulin polypeptide can be DNA or mRNA. The immunoglobulin polypeptide can include an immunoglobulin heavy chain, an immunoglobulin light chain, or both. The cryo-silicified cell and/or dehydrated cryo-silicified cell may have been prepared and/or stored described elsewhere herein.

Thus, in one aspect, this disclosure describes a method of making an antibody by expressing a polynucleotide obtained from a B cell preserved by cryo-silicification and/or cryo-silicification and dehydration. Further, this disclosure describes a method of making a library of antibodies. In this aspect, the method of making an antibody by expressing a polynucleotide from a cryo-silicified B cell is repeated, either in series or in parallel using a plurality of members of library that includes cryo-silicified B cells and/or dehydrated cryo-silicified B cells. The library of B cells used to prepare a library of antibodies may originate from a single subject or may be a combination of B cells obtained from more than one subject. Further, the various expression constructs containing the various polynucleotides can be expressed simultaneously or sequentially. Also, the various expression constructs containing the various polynucleotides can be expressed separately (e.g., in a homogeneous culture), expressed together (e.g., in a heterogeneous culture), expressed in a cell-based expression system, or expressed in a cell-free expression system.

The antibodies may be further refined. For example, stabilizing, specificity enhancing, and/or selectivity enhancing mutations may be introduced into the antibody. Additionally, tags that allow for purification, cellular localization, and/or cellular uptake may be added to the antibody.

In some embodiments where a library of antibodies is produced, the library of antibodies may serve as the base library for an antibody discovery program. For example, the library of antibodies may be base library for yeast display and/or phage display campaigns to uncover highly potent and/or selective antibodies.

The antibodies produced from an expression construct that includes a polynucleotide extracted from a cryo-silicified B cell and/or dehydrated cryo-silicified cell may be administered to a subject having, or at risk of having, a clinical condition treatable with the antibodies so produced. Thus, in another aspect, this disclosure describes a method includes treating a subject having, or at risk of having, a clinical condition. The method includes administering a composition that includes the antibodies to a subject having, or at risk of having, a particular condition in an amount effective to treat the condition. In this aspect, an “effective amount” is an amount effective to reduce, limit progression, ameliorate, or resolve, to any extent, a symptom or clinical sign related to the condition.

Treating a condition can be prophylactic or, alternatively, can be initiated after the subject exhibits one or more symptoms or clinical signs of the condition. Treatment that is prophylactic—e.g., initiated before a subject manifests a symptom or clinical sign of the condition such as, for example, while an infection remains subclinical—is referred to herein as treatment of a subject that is “at risk” of having the condition. As used herein, the term “at risk” refers to a subject that may or may not actually possess the described risk. Thus, for example, a subject “at risk” of infectious condition is a subject present in an area where other individuals have been identified as having the infectious condition and/or is likely to be exposed to the infectious agent even if the subject has not yet manifested any detectable indication of infection by the microbe and regardless of whether the subject may harbor a subclinical amount of the microbe.

Accordingly, a composition that includes the antibodies can be administered before, during, or after the subject first exhibits a symptom or clinical sign of the condition or, in the case of infectious conditions, before, during, or after the subject first comes in contact with the infectious agent. Treatment initiated before the subject first exhibits a symptom or clinical sign associated with the condition may result in decreasing the likelihood that the subject experiences clinical evidence of the condition compared to a subject to which the composition is not administered, decreasing the severity of symptoms and/or clinical signs of the condition, and/or completely resolving the condition. Treatment initiated after the subject first exhibits a symptom or clinical sign associated with the condition may result in decreasing the severity of symptoms and/or clinical signs of the condition compared to a subject to which the composition is not administered, and/or completely resolving the condition.

In another aspect, this disclosure describes a method of identifying mutations in a tumor cell. Generally, the method includes obtaining a tumor from a subject, cryo-silicifying to create a cryo-silicified a tumor cell creating a cryo-silicified cell and/or cryo-silicifying and dehydrating the tumor cell creating a dehydrated cryo-silicified cell, isolating at least one cellular material from the cryo-silicified tumor cell and/or dehydrated cryo-silicified cell, and analyzing the cellular material for differences in the cellular material of the cryo-silicified tumor cell and/or dehydrated cryo-silicified tumor cell compared to the cellular material from a normal reference cell. In some embodiments, where the tumor cell is cryo-silicified and dehydrated, the method may further include rehydrating the dehydrated cryo-silicified cell as described elsewhere herein.

The cellular material analyzed for difference can include genetic cellular material (e.g., DNA) or proteomic cellular material (e.g., protein). In some cases, a difference between the cellular material of the tumor cell and a normal cell may indicate that the subject from whom the tumor cell was obtained will respond to a selected therapy. In other cases, a difference between the cellular material of the tumor cell and a normal cell may indicate that the subject from whom the tumor cell was obtained will not respond to a selected therapy.

In yet another aspect, this disclosure describes a method of monitoring a subject's response to therapy for treating a medical condition. Generally, the method includes analyzing a first cellular material of a first cryo-silicified cell and/or first dehydrated cryo-silicified cell prepared from a cell obtained from a subject at a first time point for the presence of cellular material indicative of a medical condition, then obtaining a second cell from the subject after a period of therapy and analyzing the second cell for the presence of second cellular material indicative of the medical condition. The second cell may or may not be subject to cryo-silicification and/or cryo-silicification and dehydration. Thus, In some embodiments, the method further includes, obtaining a second cell, cryo-silicifying the second cell to create a second cryo-silicified cell and/or cryo-silicifying and dehydrating the second cell to create a second dehydrated cryo-silicified cell; storing the second cryo-silicified cell and/or the second dehydrated cryo-silicified cell; isolating a second cellular material; analyzing the second cellular material for a difference in the cellular material of the first cryo-silicified cell and/or first dehydrated cryo-silicified cell and the second cellular material from the second cryo-silicified and/or second dehydrated cryo-silicified cell. In some embodiments, where the second cell was cryo-silicified and dehydrated, the second dehydrated cryo-silicified cell, the method may further include rehydrating the second dehydrated cell as described elsewhere herein.

In the preceding description and following claims, the term “and/or” means one or all of the listed elements or a combination of any two or more of the listed elements; the terms “comprises,” “comprising,” and variations thereof are to be construed as open ended—i.e., additional elements or steps are optional and may or may not be present; unless otherwise specified, “a,” “an,” “the,” and “at least one” are used interchangeably and mean one or more than one; and the recitations of numerical ranges by endpoints include all numbers subsumed within that range (e.g., 1 to 5 includes 1, 1.5, 2, 2.75, 3, 3.80, 4, 5, etc.).

As used herein, the term “antibody” refers generally an immunoglobulin or a fragment thereof. Thus, as used herein, the term “antibody” encompasses not only immunoglobulins with an intact Fc region, but also antibody fragments capable of binding to a biological molecule (such as an antigen or receptor) or a portion thereof, including but not limited to Fab, Fab′ and F(ab′)₂, pFc′, Fd, a single domain antibody (sdAb), a variable fragment (Fv), a single-chain variable fragment (scFv) or a disulfide-linked Fv (sdFv); a diabody or a bivalent diabody; a linear antibody; a single-chain antibody molecule; and a multispecific antibody (e.g., a tribody) formed from antibody fragments. The antibody can be of any type (e.g., IgG, IgE, IgM, IgD, IgA and IgY), class (e.g., IgG1, IgG2, IgG3, IgG4, IgA1 and IgA2), or subclass.

Reference throughout this specification to “one embodiment,” “an embodiment,” “certain embodiments,” “in some embodiments,” or “some embodiments,” etc., means that a particular feature, configuration, composition, or characteristic described in connection with the embodiment is included in at least one embodiment of the disclosure. Thus, the appearances of such phrases in various places throughout this specification are not necessarily referring to the same embodiment of the disclosure. Furthermore, particular embodiments may be described in isolation for clarity. Thus, unless otherwise expressly specified that the features of a particular embodiment are incompatible with the features of another embodiment, the particular features, configurations, compositions, or characteristics may be combined in any suitable manner in one or more embodiments. Thus, features described in the context of one embodiment may be combined with features described in the context of a different embodiment except where the features are necessarily mutually exclusive.

The term “antigen” refers to any substance that is capable of being the target of an immune response. An antigen may be the target of, for example, a cell-mediated and/or humoral immune response raised by a subject organism. Alternatively, an antigen may be the target of a cellular immune response (e.g., immune cell maturation, production of cytokines, production of antibodies, etc.) when contacted with immune cells.

As used herein, the term “nucleic acid” or “oligonucleotide” refers to polynucleotides such as deoxyribonucleic acid (DNA) or ribonucleic acid (RNA). Nucleic acids include but are not limited to genomic DNA, cDNA, mRNA, iRNA, miRNA, tRNA, ncRNA, rRNA, and recombinantly produced and chemically synthesized molecules such as aptamers, plasmids, anti-sense DNA strands, shRNA, ribozymes, nucleic acids conjugates, and oligonucleotides. A nucleic acid may be single-stranded, double-stranded, linear, or covalently circularly closed molecule. A nucleic acid can be isolated. The term “isolated nucleic acid” means that the nucleic acid (i) was amplified in vitro, for example via polymerase chain reaction (PCR), (ii) was produced recombinantly by cloning, (iii) was purified, for example, by cleavage and separation by gel electrophoresis, (iv) was synthesized, for example, by chemical synthesis, or (vi) extracted from a sample. A nucleic acid might be introduced—i.e., transfected—into cells. When RNA is used to transfect cells, the RNA may be modified by stabilizing modifications, capping, or polyadenylation.

Generally, nucleic acids can be extracted, isolated, amplified, or analyzed by a variety of techniques such as those described by Green and Sambrook, Molecular Cloning: A Laboratory Manual (Fourth Edition), Cold Spring Harbor Laboratory Press, Woodbury, NY 2,028 pages (2012); or as described in U.S. Pat. Nos. 7,957,913; 7,776,616; 5,234,809; and 9,012,208. Examples of nucleic acid analysis include, but are not limited to, sequencing and DNA-protein interaction. Sequencing may be by any method known in the art. DNA sequencing techniques include classic dideoxy sequencing reactions (Sanger method) using labeled terminators or primers and gel separation in slab or capillary, and next generation sequencing methods such as sequencing by synthesis using reversibly terminated labeled nucleotides, pyrosequencing, 454 sequencing, Illumina/Solexa sequencing, allele specific hybridization to a library of labeled oligonucleotide probes, sequencing by synthesis using allele specific hybridization to a library of labeled clones that is followed by ligation, real time monitoring of the incorporation of labeled nucleotides during a polymerization step, polony sequencing, and SOLID sequencing. Separated molecules may be sequenced by sequential or single extension reactions using polymerases or ligases as well as by single or sequential differential hybridizations with libraries of probes.

As used herein “amplified DNA”, “amplified RNA” or “PCR product” refers to an amplified fragment of DNA or RNA of defined size. Various techniques are available and well known in the art to detect PCR products. PCR product detection methods include, but are not restricted to, gel electrophoresis using agarose or polyacrylamide gel and adding ethidium bromide staining (a DNA and RNA intercalant), labeled probes (radioactive or non-radioactive labels, southern blotting), labeled deoxyribonucleotides (for the direct incorporation of radioactive or non-radioactive labels) or silver staining for the direct visualization of the amplified PCR products; restriction endonuclease digestion, which relies on agarose gel electrophoresis, polyacrylamide gel electrophoresis, or high-performance liquid chromatography (HPLC); dot blots, using the hybridization of the amplified DNA or RNA on specific labeled probes (radioactive or non-radioactive labels); high-pressure liquid chromatography using ultraviolet detection; electro-chemiluminescence coupled with voltage-initiated chemical reaction/photon detection; and direct sequencing using radioactive or fluorescently labeled deoxyribonucleotides for the determination of the precise order of nucleotides with a DNA fragment of interest, oligo ligation assay (OLA), PCR, qPCR, DNA sequencing, fluorescence, gel electrophoresis, magnetic beads, allele specific primer extension (ASPE) and/or direct hybridization.

In the preceding description, particular embodiments may be described in isolation for clarity. Unless otherwise expressly specified that the features of a particular embodiment are incompatible with the features of another embodiment, certain some embodiments can include a combination of compatible features described herein in connection with one or more embodiments.

For any method disclosed herein that includes discrete steps, the steps may be conducted in any feasible order. And, as appropriate, any combination of two or more steps may be conducted simultaneously.

The present invention is illustrated by the following examples. It is to be understood that the particular examples, materials, amounts, and procedures are to be interpreted broadly in accordance with the scope and spirit of the invention as set forth herein.

### EXAMPLES

### Example 1

Cells that were cryo-silicified or cryo-silicified and dehydrated were assessed for RNA integrity.

Murine BRCA1-deficient BR5-Akt cells (3×10⁶) were washed with PBS, followed by physiological saline (154 mM NaCl), and then suspended in 1 mL silicic acid solution containing 10 mM TMOS, 100 mM NaCl, and 1.0 mM HCl (pH 3.0), with scale up as needed. Following a 5-10-minute incubation at room temperature, the cell suspension was transferred to −80° C. for cryo-silicification/storage.

Silicified cells were thawed in room temperature water, aliquoted, and centrifuged at 1200 RPM for five minutes. The supernatant was removed and cells were either directly lysed (silicified only), or dehydrated (silicified and dehydrated) overnight using a desiccator (SECADOR, Bel-Art Products Corp., Wayne, NJ), followed by lysis the next day. An RNA isolation kit (PURELINK RNA Mini Kit, Invitrogen, (Carlsbad, CA) was used to purify RNA using an 18G needle and syringe (eight times) for homogenization. Cells were directly analyzed for purity or stored at −80° C. until analysis.

A 2100 Bioanalyzer with the RNA 6000 Nano kit (Agilent, Santa Clara, CA) was used to analyze RNA integrity. Silicified or silicified/dehydrated RNA samples were run in duplicate by the University of New Mexico Comprehensive Cancer Center Analytical and Translational Genomics Shared Resource.

FIGS. 1-4 show the results. Both cryo-silicified cells and cryo-silicified dehydrated cells stored at −80° C. for more than one year retained integrity of RNA extracted from the cells (FIGS. 1, 2, 3, and 4).

Using a similar method, a unique cryo-silicified cell sample that was stored at −80° C. for 14 months was dehydrated using a SAVANT DNA 120 SPEEDVAC Concentrator (available from Thermo Fischer Scientific, Waltham, MA) at 43° C. for 30 minutes, followed by room temperature storage for 24 hours in an electronic desiccator (Si DV). The electropherogram presented in FIG. 5. A RIN score of 8.4 was calculated. The rRNA ratio (28S/18S) was 3.0 with a concentration of 764 ng/μl.

### Example 2

RNA isolated form cryo-silicified or dehydrated cryo-silicified cells was used for gene amplification.

BR5-Akt ovarian cancer cells at a density of 3×10⁶ cells/mL were washed in PBS and then isotonic saline, followed by incubation in 1 mL silicic acid solution containing in 10 mM TMOS in 100 mM NaCl, pH 3.0, for 10 minutes followed by incubation at −80° C. for at least 24 hours.

Following cryo-silicification, some cells were subsequently dehydrated. Cell dehydration was performed using the SAVANT DNA 120 SPEEDVAC Concentrator (available from Thermo Fischer Scientific). 2×10⁶ silicified cells were washed in water then suspended in 50 μL of PBS and dehydrated for 30 minutes at medium heat setting (43° C.). The resulting dry pellet was then stored in a SECADOR Vertical Desiccator Cabinet overnight (available from Thomas Scientific, Logan Township, NJ).

Fresh ID8 cells not subjected to cryo-vilification or dehydration were used as a control.

Fresh ID8 cells, cryo-silicified BR5-Akt cells, and dehydrated cryo-silicified/dehydrated BR5-Akt cells were lysed using the QIASHREDDER (available from Qiagen located in Germantown, MD) and ACCUPSIN Micro 17 microcentrifuge (available from Thermo Fisher Scientific). RNA extraction was performed using the Invitrogen PURELINK RNA Mini Kit (available from Invitrogen located in Carlsbad, CA) according to the manufacturer's protocol.

Reverse transcription was performed using SUPERSCRIPT III First-Strand Synthesis SUPERMix using the manufacturer's protocol (available from Thermo Fisher Scientific). To micrograms of RNA was used with 50 ng/μL random hexamers reverse primers (controls) or 2 μM of the BRCA1 reverse primer. RT-PCR was performed using an Eppendorf MASTERCYCLER PRO (available from Thermo Fischer Scientific).

Each PCR reaction was performed using 5 μL of 5× Colorless GoTaq Reaction Buffer (available from Promega, Madison, WI), 0.5 μL of 10 mM dNTPs (available from Thermo Fisher Scientific), 0.5 μL of 10 mM specific forward primer (available from Sigma-Aldrich, St. Louis, MO), 5 μL of 10 mM specific reverse primer, 0.25 μL GoTaq DNA Polymerase (available from Promega), 6 μL cDNA, and water to bring the volume to 25 μL.

A gel (E-Gel EX 1% Agarose, available from Invitrogen, Carlsbad, CA) was run using 10 μL of the PCR product and Tris-EDTA buffer up to 20 μL per well for 10 minutes. A 1 Kb plus DNA ladder was included and the gel was imaged using the Protein Simple FLUORCHEM R.

The results are shown in FIG. 6 and indicate that RNA isolated from both cryo-silicified cells and dehydrated cryo-silicified cells was successfully amplified.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

What is claimed is:

1. A library composition comprising:
a plurality of cryo-silicified cells, a plurality of dehydrated cryo-silicified cells, or both, wherein at least a portion of the cryo-silicified cells, the dehydrated cryo-silicified cells, or both comprises accessible biological information.

2. The library composition of claim 1, wherein the biological information comprises genetic information, proteomic information, or transcriptomic information.

3. The library composition of claim 1, wherein the cryo-silicified cells, the dehydrated cryo-silicified cells, or both are prepared from cells isolated from a patient infected with a virus, diagnosed as having a disease, or both.

4. The library composition of claim 1, wherein the cryo-silicified cells or dehydrated cryo-silicified cells are prepared from tumor cells, B cells, tissue cells, or combinations thereof.

5. A method comprising:
obtaining a sample comprising a cell;
cryo-silicifying the sample to create a cryo-silicified sample.

6. The method of claim 5, further comprising dehydrating the cryo-silicified sample to create a dehydrated cryo-silicified sample.

7. The method of claim 6, further comprising storing the cryo-silicified sample.

8. The method of claim 5, wherein the cryo-silicified sample comprises a cellular material and the method further comprising analyzing the cellular material.

9. The method of claim 8, wherein the cellular material comprises RNA, DNA, a protein, a fragment thereof, or combinations thereof.

10. The method of claim 8, wherein analyzing the cellular material comprises isolating the cellular material from the cryo-silicified sample.

11. The method of claim 8, wherein the sample comprises a cell infected with a pathogen.

12. The method of claim 11, wherein the pathogen comprises a virus comprising a viral genome, the cellular material comprises RNA fragments of the transcribed viral genome, and the method further comprises sequencing the RNA fragments and reconstructing the viral genome.

13. The method of claim 8, wherein the sample comprises a B cell, the cellular material comprises RNA, and the method further comprises:
making cDNA from the RNA;
cloning the cDNA to into an expression vector; and
expressing the expression vector to produce an immunoglobulin polypeptide encoded by the cDNA.

14. The method of claim 13, further comprising administering the immunoglobulin polypeptide to a patient.

15. The method of claim 8, wherein the cellular material comprises at least a portion of a cellular genome and the at least a portion of the cellular genome is analyzed for a genetic mutation.

16. A method comprising:
obtaining a first cell from a subject;
cryo-silicifying the first cell create a cryo-silicified cell;
storing the cryo-silicified cell;
isolating a first cellular material from the cryo-silicified cell;
obtaining a second cell;
isolating a second cellular material from the second cell; and
analyzing the first cellular material and the second cellular material for a difference between the first cellular material and the second cellular material.

17. The method of claim 16, further comprising dehydrating the cryo-silicified cell to create a dehydrated cryo-silicified cell and rehydrating the dehydrated cryo-silicified cell.

18. The method of claim 16, wherein the first cell is a tumor cell, the second cell is normal reference cell, and the difference between the first cellular material and the second cellular material indicates the subject's response to a selected therapy.

19. The method of claim 16, wherein:
the first cell is obtained from a subject inflicted with a medical condition at a first time;
the second cell is obtained from the subject at a second time point occurring at a predetermined time after a therapy is administered to the subject; and
wherein analyzing the first cellular material and the second cellular material for a difference between the first cellular material and the second cellular material comprises identifying a change in the second cellular material that is indicative of the medical condition compared to the first cellular material that is indicative of the medical condition.

20. The method of claim 17, wherein analyzing the cellular material of the second cell further comprises:
cryo-silicifying the second cell to create a second cryo-silicified cell; and
storing the second cryo-silicified cell.
